(19) United States
(12) Patent Application Publication
(10) Pub. No.: US 2024/0156394 A1
(43) Pub. Date: May 16, 2024

(54) Breastfeeding Monitor and Smart Insight System

(71) Applicant: BIB HEALTH & TECHNOLOGIES, LLC, Dover, DE (US)

(72) Inventors: PAUL GORDON, DOVER, DE (US); DAN YRIGOYEN, DOVER, DE (US); RYAN LINDSLEY, DOVER, DE (US)

(73) Assignee: BIB HEALTH & TECHNOLOGIES, LLC, Dover, DE (US)

(21) Appl. No.: 18/389,186

(22) Filed: Nov. 13, 2023

Related U.S. Application Data
(60) Provisional application No. 63/424,188, filed on Nov. 10, 2022.

(51) Int. Cl. A61B 5/00; G16H 50/20

(52) U.S. Cl. CPC A61B 5/4205; A61B 5/0002; A61B 5/6838; A61B 5/7221; A61B 5/7271; A61B 5/742; G16H 50/20; A61B 2560/045; A61B 2560/0462; A61B 2562/0204; A61B 2562/0219

(57) ABSTRACT

A system is disclosed which includes a sensor clip, having at least one sensor, and a mobile device. The mobile device wirelessly receives sensor data from the sensor clip sensor. Additionally disclosed herein is a system which includes a sensor clip, having at least one sensor, a mobile device, and a server. The mobile device wirelessly receives sensor data from the sensor clip sensor, and the server wirelessly receives sensor data from the mobile device.

### TECHNICAL FIELD

This disclosure relates generally to a breastfeeding monitor and smart insight system. More specifically, the breastfeeding monitor and smart insight system disclosed herein provides women a convenient device that may be utilized during breastfeeding to aid women in tracking parameters useful in assessing child health, growth, and development by calculating an estimated milk volume consumed and using other physiological inputs to derive smart insights.

### BACKGROUND

Both the American Academy of Pediatrics and the World Health Organization recommend breastfeeding as the exclusive means of feeding a child for the first 6 months and recommend continued breastfeeding for up to 2 years of age or longer. Additionally, the U.S. dietary Guidelines for Americans, released by the Departments of Agriculture and Health and Human Services in step with developments in nutrition science, public health, and best practices in scientific review and guidance development, recommends continuing breastfeeding after 6 months until a child is 12 months or older. According to the Center for Disease Control (“CDC”), breastfeeding is the best source of nutrition for most babies.

As a baby grows, the mother's breast milk changes to meet the child's nutritional needs. Breastfeeding can also help protect a baby against some short and long-term illnesses and diseases. For example, babies who are breastfed have a lower risk of asthma, obesity, type 1 diabetes, severe lower respiratory disease, acute otitis media (ear infections), sudden infant death syndrome (“SIDS”), and gastrointestinal infections (diarrhea/vomiting), according to the CDC. According to some relevant authorities, the nutrients of breastmilk, such as carbohydrates and protein are better absorbed and used by a baby compared with formula. Additionally, the nutrients of breastmilk are best for the growth and development of a baby's nervous system and brain. Further, a breastfed baby's eyes work better, likely because of the types of fat contained in breastmilk.

Challenges to breastfeeding include sore nipples, engorged breasts, mastitis, leaking milk, pain, failure to latch on by the infant, and low milk production. Among these challenges low milk production can be challenging to identify during breastfeeding and can be detrimental to a child's nutrition, and therefore growth and development. Often low milk consumption is not detected except by signs of the detrimental effect to the child such as a child's slow weight gain, concentrated urine, and dark, dry stools. It is, therefore, better whenever possible to track a baby's milk intake as breastfeeding progresses rather than having to rely on these detrimental effects as an indicator of insufficient milk intake during breastfeeding.

Conventionally, tracking how much breastmilk a baby is getting can be challenging and unreliable. To track how much breastmilk a baby is getting, it is often recommended to utilize a weighted feed method. In a weighted feed, the baby is weighed before and after breastfeeding to ascertain how much milk the baby is getting during a typical feed. The mother is then encouraged to continue this method and create a chart to track the amount of milk consumed at each feed. It is, however, impractical to use the weighted feed method for every feed given the constraints on time, attention, and resources that many mothers have when caring for a baby. Further, babies are oftentimes breastfed away from home and mothers cannot carry a scale everywhere they go and many scales are inconsistent, unreliable, and lack sufficient sensitivity to accurately measure a weight of a baby. Finally, even when a mother is able to frequently use the weighted feed method and diligently tracks the data, she must then consult a healthcare professional for the optimum amount her baby should be receiving and manually compare this with the data she has recorded. Thus, there is a need for a more efficient, automated, and accessible way to track the amount of food a baby is consuming at each feeding session and over the course of breastfeeding.

### SUMMARY

A system is disclosed which includes a sensor clip, having at least one sensor, and a mobile device. The mobile device wirelessly receives sensor data from the sensor clip sensor. Additionally disclosed herein is a system which includes a sensor clip, having at least one sensor, a mobile device, and a server processor. The mobile device wirelessly receives sensor data from the sensor clip sensor, and the server wirelessly receives sensor data from the mobile device.

### BRIEF DESCRIPTION OF THE DRAWINGS

Non-limiting and non-exhaustive implementations of the present disclosure are described with reference to the following figures, wherein like or similar reference numerals refer to like or similar parts throughout the various views unless otherwise specified. Advantages of the present disclosure will become better understood with regard to the following description, and accompanying drawings:

FIG. 1 illustrates an exemplary system including a breastfeeding monitor and smart insight devices.

FIG. 2A illustrates a right-side perspective view of an exemplary sensor clip.

FIG. 2B illustrates a top-right perspective view of an exemplary sensor clip.

FIG. 3 illustrates an exemplary subsystem of a sensor clip, shown as a block diagram, and a mobile device.

FIG. 4 illustrates an exemplary mobile device and server subsystem.

### DETAILED DESCRIPTION

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same or similar reference numbers are used throughout the drawings to refer to the same or similar parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

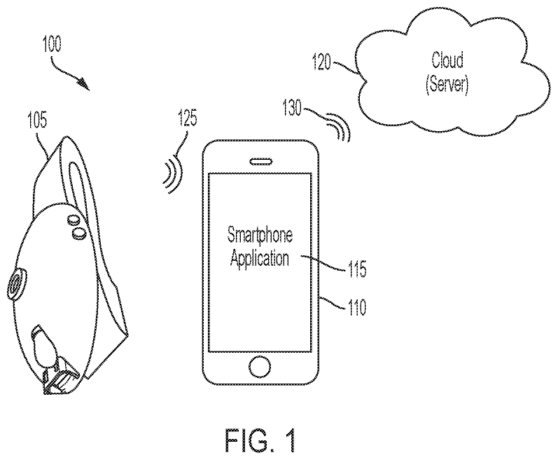

FIG. 1 illustrates an exemplary breastfeeding monitor and smart insight system. The breastfeeding monitor and smart insight system 100 may include a sensor clip 105, a mobile device 110, a network interface connection 130, and a server 120. Sensor clip 105 may include wireless communication circuitry 125. Mobile device 110 may include a smartphone application 115.

Sensor clip 105 may be designed to attach to an article of clothing, a blanket, or other item connected to mother or baby or surrounding area such that sensor clip 105 may be maintained near a user and child during breastfeeding as discussed in further detail below. Sensor clip 105 may include a variety of components that enable the device to track various feeding data. For example, sensor clip 105 may include a microphone that may be capable of detecting the sound of a child swallowing during feeding, an accelerometer for assessing movement, and a processor for processing of microphone data as discussed in further detail below.

Because sensor clip 105 is intended to be placed near a child during breastfeeding, such as being placed beneath a woman's outer clothing, a blanket, or other item, sensor clip 105 may be beyond reach with limited hand movement available during breastfeeding. Also, any user interface integrated as a part of sensor clip 105 itself may be covered by clothing, a blanket, or other item during breast feeding which may cause a user difficulty in accessing sensor clip 105 to monitor the progress of feeding, to change settings of sensor clip 105, or to otherwise interact with sensor clip 105. Therefore, mobile device 110 may be provided within the breastfeeding monitor and smart insight system 100 as a solution to allow a user to interact with sensor clip 105 more easily while sensor clip 105 is in use during a feeding session, and to access information collected by sensor clip 105 during a feeding session in real time.

For example, mobile device 110 may be implemented as a smart phone, a tablet, a laptop computer, a desktop computer, a music storage and playback device, a personal digital assistant, or any other device capable of implementing a software application that may interact with, control, and provide information from sensor clip 105. These exemplary devices may include a combination of one or more application programs and one or more hardware components. For example, application programs may include software modules, sequences of instructions, routines, data structures, display interfaces, and other types of structures that execute operation. Further, hardware components implementing modules and other means disclosed herein may include a combination of processors, microcontrollers, busses, volatile and non-volatile memory devices, non-transitory computer readable memory device and media, data processors, control devices, transmitters, receivers, antennas, transceivers, input devices, output devices, network interface devices, and other types of components that are apparent to those skilled in the art. While examples herein use a smart phone as an exemplary mobile device 110 for controlling, interacting with, and receiving information from sensor clip 105, any device capable of executing an application program may be similarly used in place of a smart phone.

Mobile device 110 may interface with sensors of sensor clip 105 using, for example, a Bluetooth low energy radio connection. Accordingly, mobile device 110 may receive data in real-time from sensors and other components of sensor clip 105, such as sounds detected by a microphone and an accelerometer signal. As information is received from the sensors of sensor clip 105, the data may be viewed in real-time, near real-time, or afterwards via a user interface of mobile device 110 either directly from sensor clip 105 or after being sent to and returned from server 120 via the network interface connection 130 of mobile device 110 with server 120 as described in further detail below. Data displayed on the user interface of mobile device 110 may be updated in real-time, for example, by updating the interface at regular and frequent intervals, such as every second or other interval known to a person having ordinary skill in the art.

A software application, for example, smartphone application 115, operating on mobile device 110 may provide further analysis of sensor data received from sensor clip 105 for display on mobile device 110. For example, a processor associated with mobile device 110 may analyze data received from sensor clip 105 to calculate and display a volume of milk consumed per feeding session based on interaction with server 120. Additionally, a processor executing smartphone application 115 may receive data analyzed by server 120 for display on mobile device 110. For example, mobile device 110 may send sensor data from sensor clip 105 to server 120 to display a calculated volume of milk consumed per feeding session as discussed in further detail below, or may itself multiply the number of detected suckling and swallowing sounds by an average volume of milk expressed per swallow or per suckle to obtain a volume of milk consumed in real time and per feeding session.

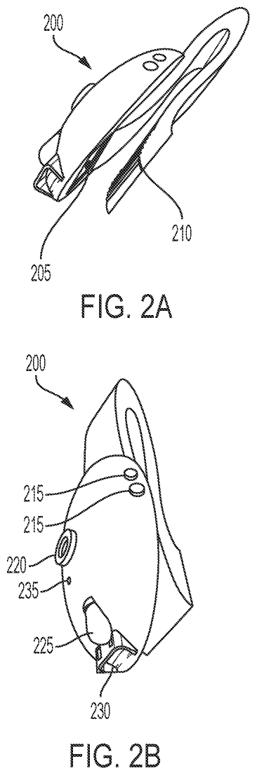

FIG. 2A illustrates a right-side perspective view of an exemplary sensor clip. Sensor clip 200 may include an electronics cavity 205, grip teeth on clip 210, control buttons 215, a camera 220, a light 225, a power port 230, microphone 235, and wireless communication circuitry 125, each of which may be similar in implementation and description to corresponding elements of FIG. 1.

In one embodiment, sensor clip 200 may include electronics cavity 205. Electronics cavity 205 may house electronic components of sensor clip 200 such as sensors, wiring, chips, batteries, and other electronics as disclosed herein. Electronics cavity 205 may be located beneath a front end of sensor clip 200 as shown in FIG. 2A, or electronics cavity 205 may be located in another location such as, in a middle section, or a back end of sensor clip 200.

In one embodiment, sensor clip 200 may be designed to clip by clip 210 to an article of clothing, a blanket, or other item, as shown in FIG. 2A, such that sensor clip 105 may be maintained near a child during breastfeeding. For example, sensor clip 200 includes an upper portion and a lower portion partially separated by a cavity where an item, such as an article of clothing or a blanket may fit to hold sensor clip 200 in place near a child during feeding. Sensor clip 200 may include grip teeth on the lower portion of clip 210 to enhance the grip of clip 210 on the item such as an article of clothing or blanket to ensure that sensor clip 200 remains in place even if jostling occurs while in use. In some embodiments, a clip mechanism for holding sensor clip 200 in place may provide an optimal functioning of sensors, such as an optimal sound signature of microphone 235 and other devices such as an accelerometer, a thermometer, and a barometer.

In some embodiments, sensor clip 200 may use other means of remaining near a child during breastfeeding. For example, sensor clip 200 may have an attached cord that may be looped around a mother's neck to allow sensor clip 200 to hang from the mother's neck, thereby holding sensor clip 200 near a child during feeding. In some embodiments, sensor clip 200 may be provided with an adhesive, or a pad with an adhesive connectable to sensor clip 200 to allow sensor clip 200 to be adhered to the mother's skin and placed adjacent to a child during feeding. Other mechanisms for maintaining sensor clip 200 near the child during feeding may be apparent to a person having ordinary skill in the art and are within the scope of this disclosure.

FIG. 2B illustrates a top-right perspective view of an exemplary sensor clip. Sensor clip 200 may include an electronics cavity 205, grip teeth 210, control buttons 215, a camera 220, a light 225, a power port 230, and wireless communication circuitry 125, each of which may be similar in implementation and description to corresponding elements of FIGS. 1 and 2A.

In some embodiments, manual control buttons 215 may be included on sensor clip 200 as shown in FIG. 2B. Control buttons 215 may each be tied to a function of sensor clip 200 as disclosed herein. For example, a manual control button may be used to start or stop a white noise generator, start or stop microphone 235 for tracking a feeding session, turn on or off light 225, change color or intensity of light 225, initiate camera 220, etc. In some embodiments, sensor clip 200 may include one or more manual control buttons. Additionally, regardless of the number of manual control buttons, control buttons 215 may be located in different areas of sensor clip 200. For example, control buttons 215 may be located on a back end and on one side of sensor clip 200 as shown in FIG. 2B or may be located in a middle section or a front end of sensor clip 200. In some embodiments, the components of sensor clip 200 which may be controlled by control buttons 215 may also be controlled by mobile device 110 through the user interface of smartphone application 115.

In some embodiments, sensor clip 200 may also include camera 220 as shown in FIG. 2B. Camera 220 may be controlled through the user interface of smartphone application 115 on mobile device 110, or alternatively may be controlled by control buttons 215. Camera 220 may allow a mother to observe a child during a feeding session without needing to physically adjust clothing or coverings by viewing the camera 220 feed on mobile device 110. Additionally, camera 220 may be used to monitor a child's crying. When crying is detected or temperature is determined to be outside a pre-determined range, a user may be alerted to check the camera 220 or to adjust the child's temperature. Camera 220 may be connectable to Wi-Fi to facilitate some functions of camera 220.

In some embodiments, sensor clip 200 may also include light 225 to provide ambient lighting as shown in FIG. 2B. Light 225 may be controlled through the user interface of smartphone application 115 on mobile device 110, or alternatively may be controlled by control buttons 215. Light 225 may be a light-emitting diode (“LED”). Light 225 may have a variety of settings to allow for different colors and intensities of ambient lighting.

Sensor clip 200 may also include power port 230. In FIG. 2B, power port 230 is shown as a mini-USB power port 230. However, any type of electrical connection is suitable. Examples may include a barrel connector, USB-A, USB-B, USB-C, a lightning port, Mini-A, Mini-B, Micro-A, Micro-B, or any other connector suitable for carrying electrical power to sensor clip 200 known to a person having ordinary skill in the art. Further, power port 230 may include one or more power ports of different sizes and connections. Also, power port 230 may be placed in a variety of locations within sensor clip 200. Sensor clip 200 may include a battery, as discussed further below, that may be charged through power port 230. Thus, sensor clip 200 may be used on battery power or direct electrical power through power port 230.

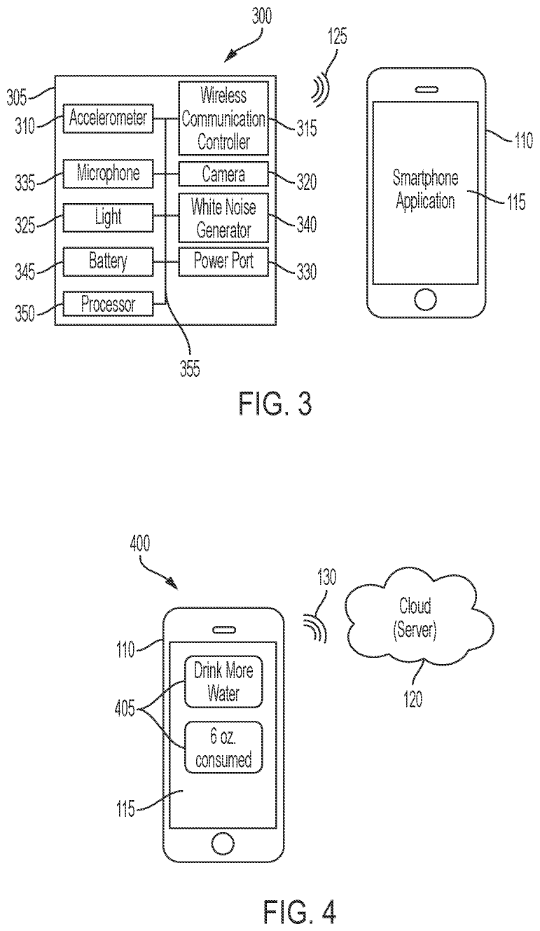

FIG. 3 illustrates an exemplary subsystem of a sensor clip, shown as a block diagram, and a mobile device. Sensor clip and mobile device subsystem 300 may include sensor clip 305, and a mobile device 110. Sensor clip 305 may include an accelerometer 310, a wireless communication controller 315, a microphone 335, a camera 320, a light 325, a white noise generator 340, a battery 345, a power port 330, a processor 350, a main bus 355, and wireless communication circuitry 125. Mobile device 110 may include a smartphone application 115 which may display smart insights as discussed in further detail below. Elements of FIG. 3 are similar in implementation and description to corresponding elements shown in FIGS. 1, 2A, and 2B.

Sensor clip 305 may have a variety of sensors and components for collecting and analyzing data. For example, microphone 335 may be used to capture sound samples during a feeding session such as the sounds of a child swallowing or a child suckling. Data captured by microphone 335 may then be transmitted by processor 350 through main bus 355 to additional components, for example, accelerometer 310. Accelerometer 310 may provide movement information for validation that a sound detected through microphone 335 was the result of a child swallowing/suckling and not merely caused by movement. Relevant sound signals captured by microphone 335 and analyzed by processor 350 include swallow/suckling patterns for a specific frequency and decibel range of swallows/suckles based on the age of a baby. For example, an infant older than six months may make swallow/suckling sounds that are between 800 hz and 1500 hz. An infant younger than six months may make swallow/suckling sounds that are between 2500 hz and 3000 hz. In any case, swallow/suckling sounds that are detected by microphone 335 may be identified by being within a predetermined frequency band between 800 hz and 3000 hz. Processor 350 may then send data collected by sensors and components such as sound samples and the accelerometer signal to wireless communication controller 315.

In one embodiment, wireless communication controller 315 may include processor 350 and establish communication with mobile device 110 by wireless communication circuitry 125. Exemplary wireless communication circuitry 125 may be implemented using Bluetooth®, Wi-Fi, ZigBee, Z-Wave, RF4CE, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, the standard formerly known as 802.15.1, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between sensor clip 305 and mobile device 110 known to one of ordinary skill in the art. Thus, wireless communication controller 315 may transmit data to mobile device 110 for further processing. Additionally, a user may control the various components of sensor clip 305 through wireless communication circuitry 125 from smartphone application 115 of mobile device 110. Further, sensor clip 305 may include personal assistant functionality which a user may access through microphone 335 to add tasks, reminders, and other items to smartphone application 115 or other applications on mobile device 110.

White noise generator 340 may help with provide an environment outside of feeding sessions where data collection is not interrupted by ambient noise. In one embodiment, certain frequencies of noise may be filtered using white noise generator 340. In other embodiments, decibel interruption may be used by cutting off the microphone in response to sounds having a certain decibel threshold (e.g., loudness). White noise generator 340 may be controlled through the user interface of smartphone application 115 on mobile device 110, or alternatively may be controlled by control buttons 215.

Battery 345 may be recharged by the user as necessary to continue to utilize sensor clip 305. Components of sensor clip 305, for example processor 350, wireless communication controller 315, and accelerometer 310, etc. may be powered by battery 345 through main bus 355. Once battery 345 has been discharged, battery 345 may be recharged by the user. To recharge battery 345, the user may connect a power source to sensor clip 305 through power port 330. Power port 330 allows power to flow from power source to battery 345 through main bus 355.

FIG. 4 illustrates an exemplary mobile device and server subsystem. Mobile device and server subsystem 400 may include a mobile device 110, a network interface connection 130, and a server 120. Mobile device 110 may include a smartphone application 115 which may display smart insights 405. Elements of FIG. 4 are similar in implementation and description to corresponding elements shown in FIGS. 1, 2A, 2B and 3.

Once mobile device 110 has received data from sensor clip 305 via wireless communication circuitry 125, mobile device 110 may aggregate and use this data to update a user interface of mobile device 110 or send data to server 120 for further processing. For example, data received by mobile device 110 such as sound samples and the accelerometer signal may be transferred to server 120 for further analysis.

As shown in FIG. 4, mobile device 110 may transmit information received from sensor clip 305 to server 120 by means of network interface connection 130. In this example, mobile device 110 implements a communication interface protocol suitable for transmission of data over large distances. For example, Network interface connection 130 may be implemented using Wi-Fi, ZigBee, Z-Wave, RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between mobile device 110 and server 120.

Server 120 may be implemented as a cloud-based server 120. For example, a cloud-based server 120 may be implemented as several servers connected in a fashion to perform server 120 functions in a partitioned series of processing steps in order to produce faster results as a function of the combined processing power of many servers working together to accomplish a particular end. Additionally, data received by server 120 may be made available via web browser by any allowed user. Accordingly, another parent, guardian, health care professional, doctor, counselor, or any other allowed person may monitor, download, and backup data in real time via the web browser. This availability of data to additional authorized users may provide a mother with added support in managing her breastfeeding when necessary. Additionally, this availability of data to authorized users also facilitates a mother's ability to communicate with authorized users remotely, such as via a remote appointment with her physician, since the physician may have a full and accurate understanding of the mother's breastfeeding data even before such an appointment.

Server 120 may analyze data received from mobile device 110. For example, server 120 may use machine learning and/or other artificial intelligence tools to identify an amount of milk consumed by a child during a breastfeeding session based on sound data, milk production data, and any other data collected by either sensor clip 305 or mobile device 110. Additionally, server 120 may utilize a variety of data sources to produce information useful to a mother throughout the breastfeeding period. For example, server 120 may compare data collected by sensor clip 305 and data generated by server 120 to provide further analysis using data such as data input by a user, such as the age, sex, weight, and other parameters of a baby and the age, weight, breast size, and other parameters of a mother. Additionally, data from medical authorities, such as suggested milk intake amounts for a baby at a given age, suggested water intake and nutrition of mother for optimal milk production and nutritional value, etc. may be used by server 120 to provide useful information. This information may then be communicated back to mobile device 110 for display on smartphone application 115 in the form of smart insights 405.

Smart insights 405 may provide information and suggestions to a mother. For example, smart insights 405 may include information such as a feeding session volume in real-time and total, an average feeding session volume over a given time, and a baby's growth and development progress through a variety of parameters. Additionally, smart insights 405 may include suggestions such as how a mother may manage milk production such as consuming more water, when to consume water, when to feed, etc. In this manner, a mother may gain the benefit of the recorded data, artificial intelligence computing, such as machine learning, and aggregated data over the course of breastfeeding to avoid any detrimental effects to, or slowed growth of, her baby. For example, a mother need not be alerted to her baby's lack of milk intake by such detrimental effects as slow weight gain, concentrated urine, and dark, dry stools.

Although the present disclosure has been described in considerable detail with reference to certain preferred versions thereof, other versions are possible. Therefore, the spirit and scope of the appended claims should not be limited to the description of the preferred versions contained therein.

What is claimed is:

1. A system, comprising:
a sensor clip device including a processor and having at least one sensor; and
a mobile device,
wherein the mobile device wirelessly receives sensor data from the at least one sensor.

2. The system of claim 1, wherein the at least one sensor is a microphone.

3. The system of claim 2, wherein the processor receives sensor data from the microphone and transmits the received sensor data to the mobile device.

4. The system of claim 3, wherein the mobile device analyzes the sensor data to identify child suckling and child swallowing sounds in a pre-determined frequency range.

5. The system of claim 4, wherein the pre-determined frequency range is between 800 hz and 1300 hz.

6. The system of claim 5, wherein the mobile device identifies a number of child suckling and swallowing sounds over a predetermined amount of time.

7. The system of claim 6, wherein the mobile device calculates a volume of milk ingested by multiplying a constant per-swallow volume by a number of identified suckling and swallowing sounds during the predetermined time.

8. The system of claim 7, wherein the mobile device displays one or more of a real time volume of swallowed milk and a volume of swallowed milk per feeding session.

9. The system of claim 1, wherein the at least one sensor includes an accelerometer and a microphone.

10. The system of claim 2, wherein the mobile device compares accelerometer data with microphone data to validate detection of a suckle or swallowing sound.

11. A system, comprising:
a sensor clip including a processor and having at least one sensor;
a mobile device; and
a server,
wherein the mobile device wirelessly receives sensor data from the at least one sensor, and the server wirelessly receives sensor data from the mobile device.

12. The system of claim 11, wherein the at least one sensor is a microphone.

13. The system of claim 12, wherein the processor receives sensor data from the microphone and transmits the received sensor data to the mobile device.

14. The system of claim 13, wherein the mobile device transmits the sensor data to the server device and the server device analyzes the sensor data to identify child suckling and child swallowing sounds in a pre-determined frequency range.

15. The system of claim 14, wherein the pre-determined frequency range is between 800 hz and 1300 hz.

16. The system of claim 15, wherein the server device applies an artificial intelligence model to identify an volume of milk ingested by a child.

17. The system of claim 16, wherein the identification of a volume of milk ingested by a child is provided on a real-time or near real-time basis.

18. The system of claim 16, wherein the identification of a volume of milk ingested by a child is provided on a per-feeding basis.

19. The system of claim 11, wherein the at least one sensor includes an accelerometer and a microphone.

20. The system of claim 19, wherein the artificial intelligence model within the server device compares accelerometer data with microphone data to validate detection of a suckle or swallowing sound.
